(19) United States
(12) Patent Application Publication
(10) Pub. No.: US 2016/0266106 A1
(43) Pub. Date: Sep. 15, 2016

(54) MULTIPLEX BIOASSAY PLATFORM USING CUT FIBER BUNDLE

(71) Applicant: SEOUL NATIONAL UNIVERSITY R&DB FOUNDATION, Seoul (KR)

(72) Inventors: Sunghoon KWON, Seoul (KR); Jiyun KIM, Seoul (KR); Sangwook BAE, Seoul (KR); Seowoo SONG, Seoul (KR)

(21) Appl. No.: 15/030,617

(22) PCT Filed: Oct. 28, 2014
(86) PCT No.: PCT/KR2014/010177

§ 371 Date: Apr. 20, 2016

(30) Foreign Application Priority Data
Oct. 28, 2013 (KR) 10-2013-0128740

(51) Int. Cl. G01N 33/543; C23C 4/14; G01N 1/36

(52) U.S. Cl. CPC G01N 33/54366; G01N 1/36; C23C 4/14

(57) ABSTRACT

The present invention proposes a new structuring method for producing slices comprising fiber fragments through a series of steps of functionalizing fibrous materials, bundling the functionalized fibrous materials, and thinly cutting the bundle. Based on the fiber bundle fragments, ultra-low cost multiplexed bioassay platforms are developed.

### TECHNICAL FIELD

The present disclosure relates to a multiplexed bioassay platform using a slice of a fiber bundle.

### BACKGROUND ART

Immunoassays are currently the most commonly used methods for the detection of proteins. Immunoassays are enzyme-linked immunosorbent assays (ELISAs) that use enzymatic signal amplification and are conducted based on 96-well plates as platforms. However, large amounts of reagents are required per assay and an operator should carry out multiple processes in each assay. That is, a large number of assays involve considerable time and cost. There is thus a need to reduce the assay time and cost in order to increase access to the detection and analysis of biomolecules. Under these circumstances, multiplexed bioassay platforms have been developed that can conduct a large number of assays on reduced amounts of samples at one time.

Big biotechnology companies as well as laboratories are currently developing a variety of platforms for multiplexed bioassays that utilize advanced technologies, such as nanotechnologies. Typical examples are bead-based assays utilizing surface-treated nanobeads, which have already been used successfully and commercialized by companies, such as Luminex and IIlumina. For cytokine profiling, different types of antibodies are attached to the surfaces of basically different beads, assays are conducted sequentially, and the assay signals are analyzed using an analytical system. Cytokine profiling requires greatly reduced amounts of samples compared to 96-well plate-based assays and uses fluorescent materials mainly as markers. Other techniques are known in which microfluidic channels surface coated with antibodies are designed such that reduced amounts of samples are used in assay processes. Various multiplexed bioassay techniques have been developed or are currently being developed that can be used to conduct a number of assays at one time, which reduces the amount of samples and time required for the assays. However, such multiplexed bioassay techniques require expensive systems and equipment operated by skilled operators for the analysis of the assay results and use very expensive raw materials for the fabrication of assay platforms, limiting their use in a wide range of applications. Thus, there have been increasing demands for multiplexed bioassay platforms in developing countries, unskilled persons, and various industrial sectors. In order to satisfy such demands and enhance access to multiplexed bioassay platforms, various requirements, such as low prices, the use of reduced amounts of samples, easy experimental processes, low analysis costs, and high scalability, need to be met. Development of scalable, inexpensive bioassay platforms that meet the above requirements is needed to keep pace with demands for the detection of biomolecules in various fields, such as human health, new drug development, and contamination analysis.

### DETAILED DESCRIPTION OF THE INVENTION

### Problems to be Solved by the Invention

An object of the present invention is to provide a multiplexed bioassay platform based on a slice including fiber fragments.

### Means for Solving the Problems

According to one aspect of the present invention, there is provided a method for fabricating bioassay platforms, including (a) coating the surfaces of fiber strands with different types of detection substances reacting specifically with target analytes to obtain reactive fiber strands, (b) collecting and structuring the reactive fiber strands coated with the detection substances to form a fiber bundle wherein the reactive fiber strands are immobilized using a matrix material in the course of the structuring, and (c) cutting the fiber bundle such that the cross-sections lie perpendicular to the lengthwise direction of the fiber bundle, to obtain slices in which fragments of the fiber strands are immobilized in the matrix material.

According to a further aspect of the present invention, there is provided a bioassay method including (a) coating the surfaces of fiber strands with different types of detection substances reacting specifically with target analytes to obtain reactive fiber strands, (b) collecting and structuring the reactive fiber strands coated with the detection substances to form a fiber bundle wherein the reactive fiber strands are immobilized using a matrix material in the course of the structuring, (c) cutting the fiber bundle such that the cross-sections lie perpendicular to the lengthwise direction of the fiber bundle, to obtain slices in which fragments of the fiber strands are immobilized in the matrix material, and (d) using the slices as bioassay platforms.

According to another aspect of the present invention, there is provided a bioassay platform including a matrix and a fiber bundle section composed of fragments of reactive fiber strands immobilized in the matrix wherein the fragments of the reactive fiber strands are coated with different types of detection substances.

### Effects of the Invention

The bioassay platform of the present invention can detect an increased number of kinds of analytes simply by increasing the number of the fiber strands bundled together in the course of structuring, indicating very high scalability of the system. In addition, the fiber strands provided with various functions do not significantly affect the overall fabrication cost of the platform because the fiber itself is very cheap and is produced without the need for any special techniques, enabling the fabrication of the platform at low cost. Furthermore, since the platform of the present invention can detect and analyze signals without a separate dedicated system, the need for an expensive analytical system is avoided, achieving low-cost analysis of the results.

### BRIEF DESCRIPTION OF THE DRAWINGS

FIG. 1 is a flowchart of a method for fabricating bioassay platforms according to one embodiment of the present invention.

FIG. 2 shows the surface modification of fiber strands by chemical treatment in accordance with one embodiment of the present invention.

FIG. 3 shows the results of bioassays obtained using single fiber strands in accordance with one embodiment of the present invention.

FIG. 4 shows fiber strands structured by various methods in accordance with exemplary embodiments of the present invention.

FIG. 5 shows a method for fabricating bioassay platforms using tape according to one embodiment of the present invention.

FIG. 6 shows a method for fabricating bioassay platforms using paraffin according to one embodiment of the present invention.

FIG. 7 shows various methods for coding fiber bundles in accordance with exemplary embodiments of the present invention.

FIG. 8 is a design of a method for fabricating bioassay platforms according to one embodiment of the present invention.

FIG. 9 is a design of a fiber bundle and a bioassay platform according to one embodiment of the present invention.

FIG. 10 is a design of a fiber bundle and a bioassay platform according to one embodiment of the present invention.

FIG. 11 shows the results of bioassays obtained using fluorescence signals.

FIG. 12 is a graph showing a change in fluorescence intensity depending on antigen concentration.

FIG. 13 shows the results of bioassays obtained using color signals.

FIG. 14 is a graph showing the intensities of color signals during bioassays.

FIG. 15 is a graph showing changes in the intensity of color signals depending on the concentrations of antigens.

FIG. 16 shows the results of multiplexed bioassays obtained using color signals.

### MODE FOR CARRYING OUT THE INVENTION

One aspect of the present invention provides a method for fabricating bioassay platforms, including (a) coating the surfaces of fiber strands with different types of detection substances reacting specifically with target analytes to obtain reactive fiber strands, (b) collecting and structuring the reactive fiber strands coated with the detection substances to form a fiber bundle wherein the reactive fiber strands are immobilized using a matrix material in the course of the structuring, and (c) cutting the fiber bundle such that the cross-sections lie perpendicular to the lengthwise direction of the fiber bundle, to obtain slices in which fragments of the fiber strands are immobilized in the matrix material.

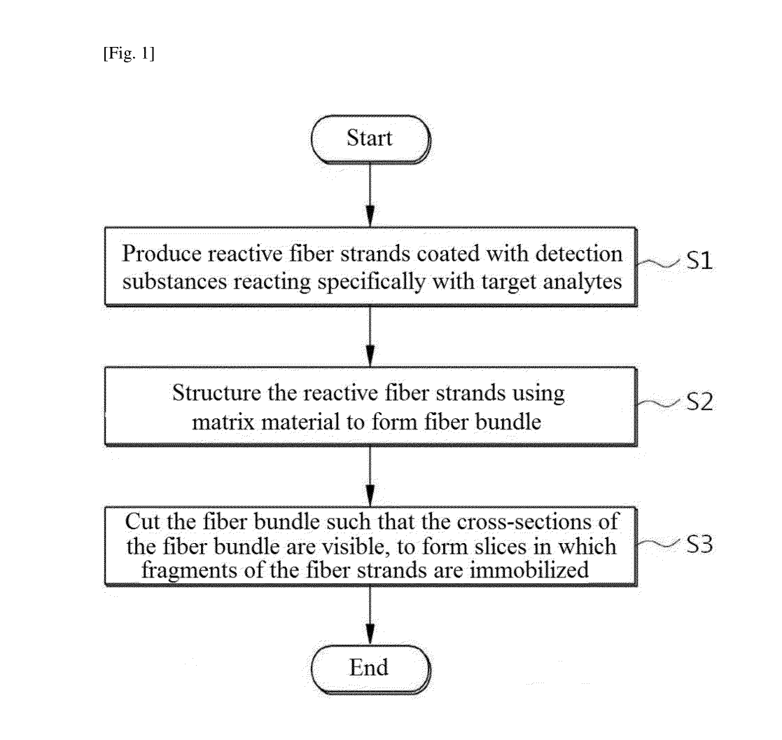

Referring first to FIG. 1, the surfaces of fiber strands are coated with different types of detection substances reacting specifically with target analytes to obtain reactive fiber strands (Si). Examples of suitable fibers include, but are not limited to, polymeric fibers (cellulose-based fibers, such as cotton fibers, natural polymeric fibers, such as collagen, and synthetic polymeric fibers, such as nylons) and carbon fibers. The fiber may include any material that is in the form of an elongated filament or thread and has the ability to adsorb or accommodate the detection substances. Preferably, the fiber is a cotton fiber that is very inexpensive, readily available, and easy to functionalize with various chemical functional groups on its surface.

There is no restriction on the dimensions (such as length and thickness) of the fiber strands used to implement the present invention. As the length of the fiber strands increases, the length of the fiber bundle increases and the number of bioassay platforms obtained after cutting of the fiber bundle increases. Accordingly, the length of the fiber strands can be adjusted depending on the desired number of the platforms. The cross-sectional size of the fiber bundle increases with increasing thickness of the fiber strands. Therefore, the thickness of the fiber strands determines the size of final bioassay platforms. However, the size of the bioassay platforms can be arbitrarily adjusted, if needed, because it has no substantial influence on the performance of the bioassay platforms. That is, since the physical features of the fiber strands do not greatly affect the performance of the bioassay platforms, the fiber may vary in thickness and length.

The detection substances reacting specifically with target analytes may be protein- or nucleic acid-based biomolecules, such as antibodies, enzymes or disease markers, and may include any substances that can be attached to the fiber strands and react with target analytes.

In one embodiment of the present invention, the detection substances may be coated by physical adsorption. The detection substances can be simply introduced into the fiber strands in such a way that the fiber strands are dipped in different solutions containing the individual detection substances, taken out from the solutions, and dried.

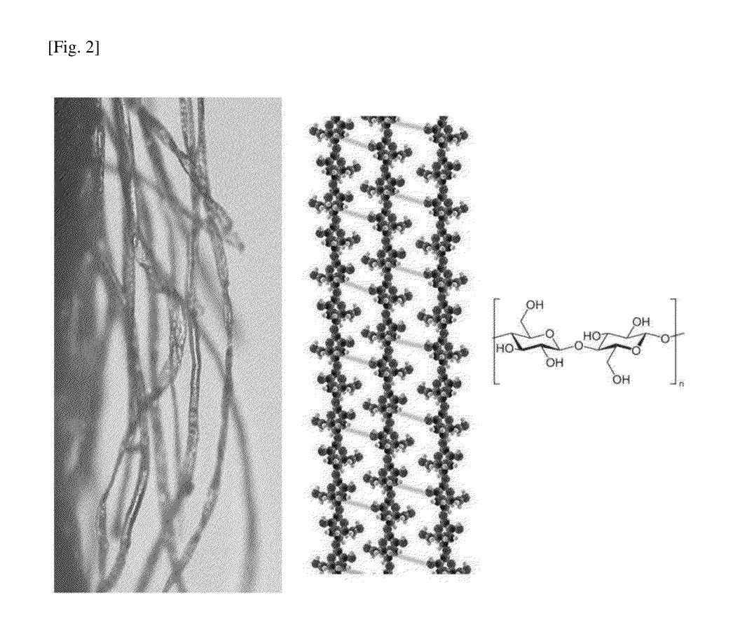

In a further embodiment of the present invention, the detection substances may be coated on the fiber strands by chemical binding. Specifically, the detection substances can be chemically linked to the fiber strands by treating the surfaces of the fiber strands with a chemical substance to form functional groups, activating the functional groups, soaking the fiber strands with different solutions containing the individual detection substances, and drying the soaked fiber strands. FIG. 2 shows the surface modification of the fiber strands by chemical treatment in accordance with one embodiment of the present invention. Referring to FIG. 2, the surface hydroxyl groups of cellulose-based fiber strands can be used to form carboxylated amine groups on the surfaces of the fiber strands. This chemical treatment enables highly efficient immobilization of the detection substances on the surfaces of the fiber strands and can increase the reactivity of the detection substances with target analytes. As a result, the intensities of detection signals for target analytes can be enhanced and the limit of detection can be lowered.

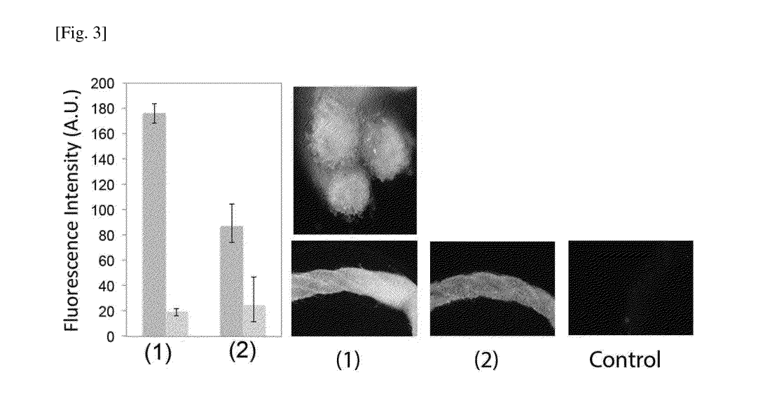

FIG. 3 shows the results of bioassays for interleukin 4 (IL4) using reactive fiber strands in accordance with one embodiment of the present invention. In FIG. 3, the left graph compares the intensity of a bioassay signal generated (1) when the detection substance is chemically linked to the single fiber strand with that generated (2) when the detection substance is adsorbed to the single fiber strand. The violet bars in the graph show the fluorescence intensity generated (1) when the detection substances are chemically linked to the fiber strands and the fluorescence intensity generated (2) when the detection substances are simply adsorbed to the fiber strands, and the yellowish green bars show the fluorescence intensities of controls untreated with the detection substances. Referring to the right image in FIG. 3, a fluorescence signal can be detected from (2) the single fiber strand adsorbed by the detection substance and a stronger signal can be detected (1) when the detection substance is chemically linked to the single fiber strand. In (1), the fluorescence signal is uniformly found over the entire area of the fiber.

Next, the reactive fiber strands coated with the detection substances are collected and structured to form a fiber bundle wherein the reactive fiber strands are immobilized using a matrix material in the course of the structuring (S2 of FIG. 1).

Each of the fiber strands may be treated with the same detection substance and the fiber strands may be treated with the same or different types of detection substances. The different types of detection substances may be bound to the fiber strands by soaking the fiber strands with different solutions containing the individual detection substances. When the functionalized fiber strands bound with the detection substances are used for bioassays, the different types of detection substances immobilized on the reactive fiber strands are physically bound to or chemically react with target analytes specifically acting on the detection substances, and as a result, the specific target analytes can be detected.

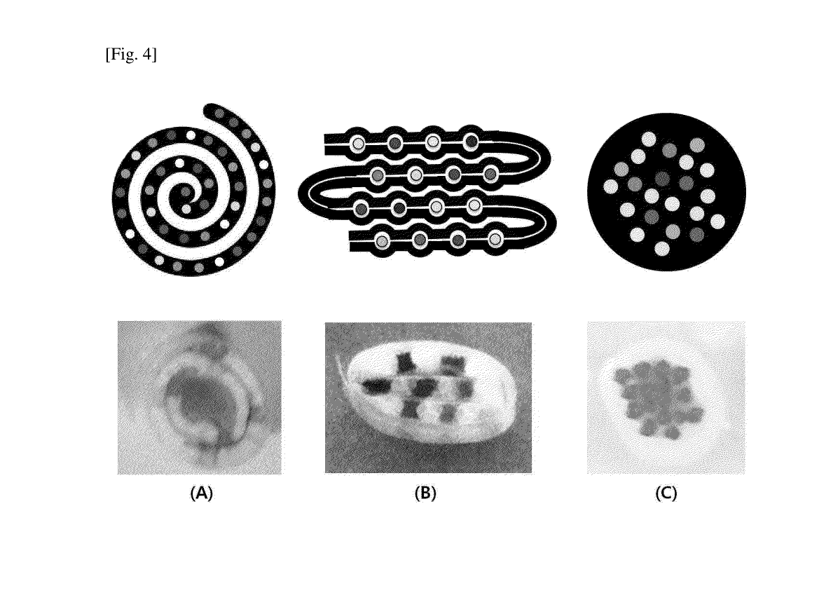

The reactive fiber strands coated with the detection substances can be collected and structured to make a fiber bundle. When the fiber strands treated with the different types of detection substances are bundled together, high scalability for multiplexed assays can be ensured. FIG. 4 shows fiber strands housed or coated by various methods in accordance with exemplary embodiments of the present invention. Referring to FIG. 4, the fiber strands are formed into a sheet, which is then rolled up (A) or folded and stacked (B) to form a bundle. Alternatively, a substance is filled between the fiber strands to fix a bundle of the fiber strands at one time, like noodle making ((C) of FIG. 4). However, the structuring of the fiber strands is not limited to the exemplary embodiments. For example, any structuring method may be used by which the fiber strands can be spatially separated from each other to prevent cross-contamination in bioassays and the types of the detection substance immobilized on the fiber strands can be identified based on the positional information of the fiber strands.

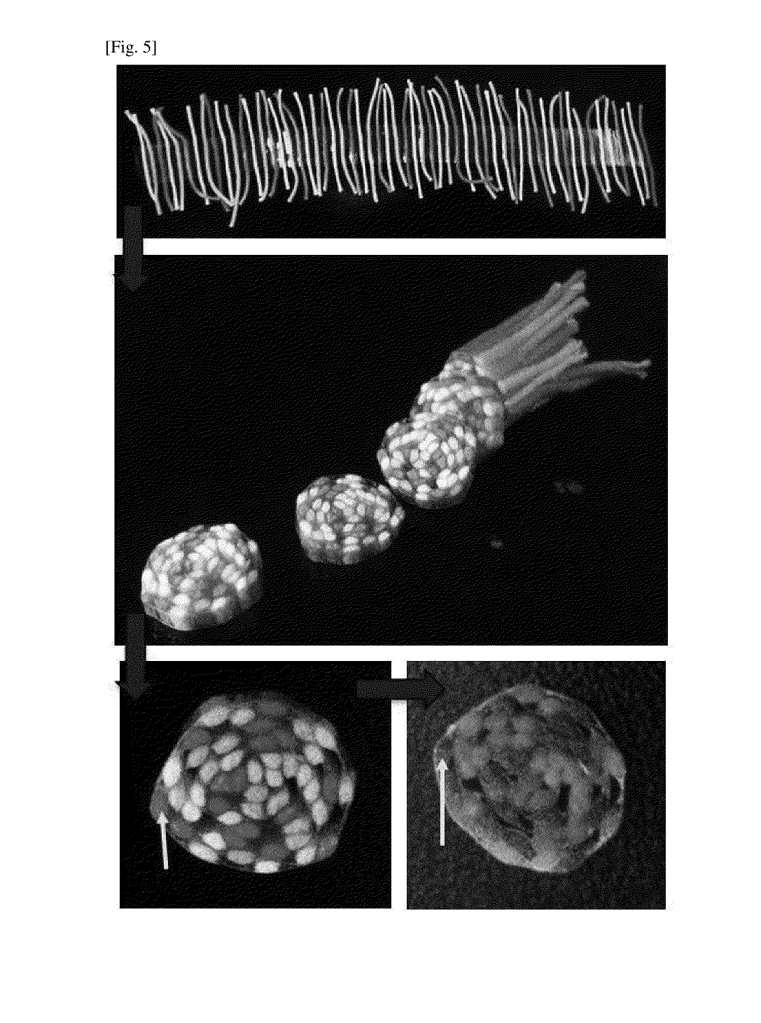

FIG. 5 shows a method for fabricating bioassay platforms by structuring fiber strands using tape to make a fiber bundle according to one embodiment of the present invention. Referring to FIG. 5, fiber strands functionalized with various substances are aligned on an adhesive film, such as tape, and covered with another layer of tape to form a thin sheet, which is then rolled up to make a bundle of the fiber strands. Instead of tape, any material capable of fixedly positioning and spatially separating the fiber strands may also be used as a matrix material for structuring the fiber strands. The structured fiber bundle is thinly sliced and the fragments can be dipped in a sample to observe changes of the fiber strands.

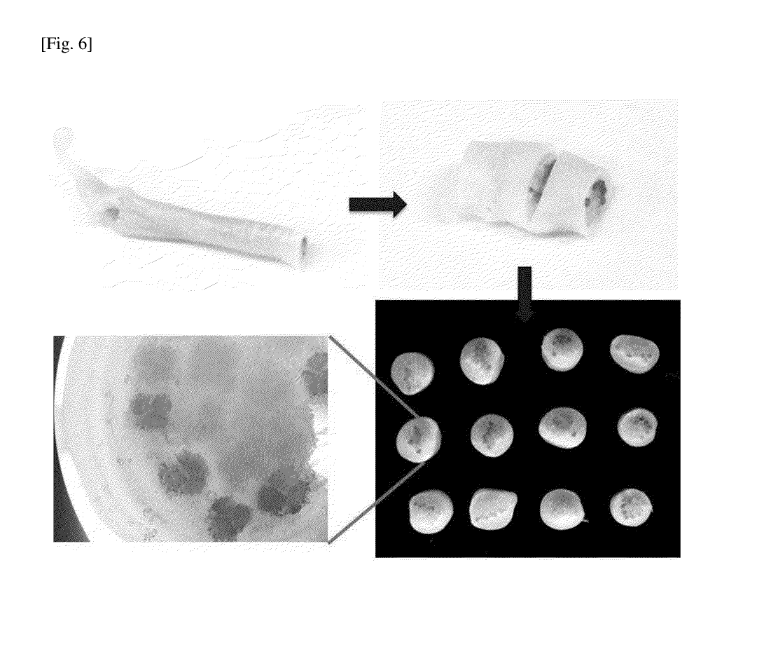

FIG. 6 shows the preparation of a fiber bundle by covering with paraffin in accordance with one embodiment of the present invention. Referring to FIG. 6, fiber strands are collected, dipped in liquid paraffin, taken out from the paraffin, and cooled to solidify the paraffin. As a result, a fiber bundle is formed. In this case, the fiber strands are completely spatially isolated from each other to prevent cross-contamination between the bundled detection regions upon subsequent bioassays. Instead of paraffin as a matrix material for covering the fiber strands, for example, a curable polymer resin may be used that per se is liquid but is slowly solidified under specified conditions. The matrix material should be chemically stable and not be so non-toxic to destroy biomolecules. The matrix material should not destroy biomolecules, such as nucleic acids, at temperatures where it is in the state of liquid, it is in the state of solid, and undergoes a phase change from liquid to solid. In a further embodiment of the present invention, the fiber strands may be structured with tape and immobilized with paraffin to make a fiber bundle.

In one embodiment of the present invention, the structuring process may further include arranging coding fiber strands to identify information of the detection substances present in the reactive fiber strands. In the structuring process, the reactive fiber strands and the coding fiber strands may be bundled together. In this case, the types of the detection substances attached to the reactive fiber strands may be identified through coding information of the coding fiber strands.

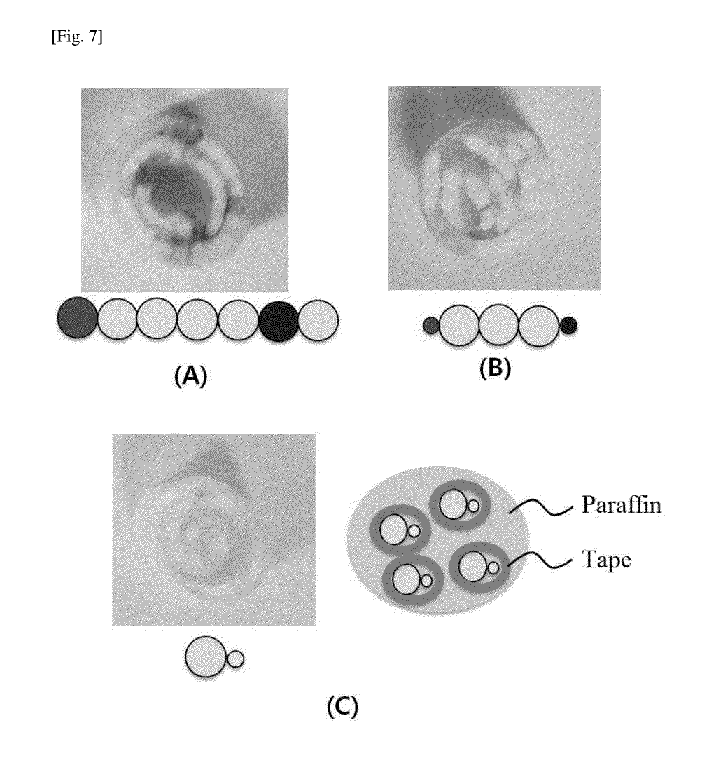

In one embodiment of the present invention, the structuring process may be performed by varying the color, number, arrangement order, size or a combination thereof of the coding fiber strands corresponding to the reactive fiber strands. FIG. 7 shows various methods for coding the fiber bundles in accordance with exemplary embodiments of the present invention. The reactive fiber strands are colored white and the coding fiber strands are colored red, blue, and yellow. Referring to FIG. 7, the reactive fiber strands and the coding fiber strands with various colors may be regularly arranged and may be structured into a fiber bundle, as shown in (A). The reactive fiber strands may be coded with different sizes of the coding fiber strands, as shown in (B). Alternatively, a single reactive fiber strand may be coupled with a single coding fiber strand and the coupled fiber strands may be structured to form a fiber bundle, as shown in (C). The reactive fiber strand and the coding fiber strand are immobilized with tape and all fiber strands are then covered with paraffin to form fiber bundles.

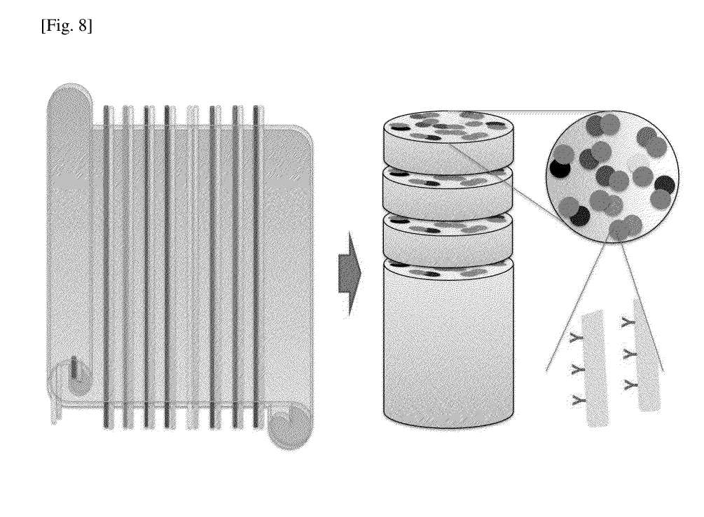

FIG. 8 is a design of a method for fabricating bioassay platforms according to one embodiment of the present invention. In FIG. 8, reactive fiber strands are represented in gray and coding fiber strands are represented in various colors. Referring to FIG. 8, the reactive fiber strands bound with various detection substances and the coding fiber strands with various colors are paired in a one-to-one relationship and then all fiber strands are collected and structured. In this case, the colors of the coding fiber strands may be used to identify the types of the detection substances present in the adjacent reactive fiber strands. The use of the coding method enables coding of various signals by varying the colors, sizes, numbers, orders, etc. of the reactive fiber strands and the coding fiber strands.

In one embodiment of the present invention, the structuring process may further include arranging magnetically controllable fiber strands into which a magnetic material is introduced.

The magnetically controllable fiber strands may be produced by chemically or physically attaching magnetic particles to the surfaces of fiber strands. The chemical method may be carried out in such a manner that chemical functional groups are attached to fiber strands, magnetic particles are surface coated with a substance capable of attaching the chemical functional groups thereto, and the two substances are chemically attached to each other. The physical method is based on physical adsorption of nano-sized particles and may be carried out in such a manner that fiber strands are simply dipped in a liquid in which magnetic particles are spread, taken out from the liquid, and dried. In both methods, various kinds of magnetic particles with various sizes, including super-paramagnetic particles, can be utilized and the amount of the magnetic particles attached to the fibers can be controlled by varying the concentration of the magnetic particles present in the liquid in the course of chemical or physical attachment. The reactive fiber strands and the magnetically controllable magnetic fiber strands are structured together so that sections of the fiber bundle can be controlled using a magnetic field or a magnet in bioassays. The fiber strands attached with a larger amount of magnetic particles respond more easily to an external magnetic field and the fiber strands attached with a smaller amount of magnetic particles respond less sensitively to an external magnetic field. Therefore, the bioassay platforms can be adjusted using an external magnetic field.

In a further embodiment of the present invention, the matrix material may include a magnetic material. In this case, magnetic particles may be mixed with liquid paraffin used in the course of structuring the fiber bundle. Then, the mixture is solidified to structure the fiber bundle. The matrix material including a magnetic material is used to structure the fiber bundle without additional magnetically controllable fiber strands so that sections of the fiber bundle can be controlled using a magnetic field or a magnet in bioassays. The response of the fiber bundle to an external magnetic field can be controlled by varying the concentration of the magnetic particles mixed with the matrix material.

Finally, the fiber bundle is cut such that the cross-sections lie perpendicular to the lengthwise direction of the fiber bundle, to obtain slices in which fragments of the fiber strands are immobilized in the matrix material (S3 of FIG. 1). Referring again to FIGS. 5 and 6, a suitable tool, such as a knife, may be used to cut the fiber bundles into fragments with predetermined thicknesses in which the cross-sections of the fiber strands constituting the fiber bundles are visible. The cross-sections of the fiber strands performing their own functions are observed in the cross-section of each slice. The slices can be used as multiplexed bioassay platforms.

A further aspect of the present invention provides a bioassay method including (a) coating the surfaces of fiber strands with different types of detection substances reacting specifically with target analytes to obtain reactive fiber strands, (b) collecting and structuring the reactive fiber strands coated with the detection substances to form a fiber bundle wherein the reactive fiber strands are immobilized using a matrix material in the course of the structuring, (c) cutting the fiber bundle such that the cross-sections lie perpendicular to the lengthwise direction of the fiber bundle, to obtain slices in which fragments of the fiber strands are immobilized in the matrix material, and (d) using the slices as bioassay platforms.

Bioassays are conducted on the sectioned fiber bundle fragments and the assay results can be evaluated through detection signals labeled on the cross-sections of the fiber bundle sections. When the platforms are treated with a sample containing target analytes, the fragments of the reactive fiber strands reacting specifically with the target analytes may be labeled with signals. Examples of methods for distinguishing different kinds of detection signals in multiplexed bioassays include, but are not limited to, utilization of positional information of the fiber strands coated with the detection substances, use of different markers, and insertion of codes into reactive sites.

In one embodiment of the present invention, when the reactive fiber strands are regularly aligned and structured into a fiber bundle, the reactive fiber strands are fixedly positioned in the fiber bundle, and as a result, the types of the coated detection substances can be identified using the positional information. In a further embodiment of the present invention, makers, such as radioactive compounds, fluorescent materials, luminescent materials, color-emitting materials, enzymes, and metals, may be linked to target analytes in the bioassays to distinguish detection signals. In another embodiment of the present invention, the reactive fiber strands are structured together with a fiber containing coding information so that information included in detection signals can be identified using the coding fiber strands to easily determine the types of the detection substances treated on the fiber strand fragments.

The platforms can be used for bioassays, such as immunoassays and enzymatic assays, to detect various substances. The method for structuring the fiber strands minimizes the volume of the platforms in which the fiber strand fragments isolated from each other gather, which reduces the amount of a sample used during the entire assay process. When the number of the reactive fiber strands bundled together in the course of the structuring for the fabrication of the platforms is increased, an increased number of kinds of target analytes can be detected, indicating very high scalability of the systems.

In order to distinguish different kinds of detection signals in the multiplexed bioassays, the cross-sections of the fiber bundle fragments are observed visually or by microscopy or are imaged using a suitable device, such as a camera, and signal-labeled portions and coding information are read from the image to analyze the assay results. In one embodiment of the present invention, fluorescence signals or color changes as the detection signals from the reactive fiber fragments may be observed to determine whether reactions occur.

An analytical system is used to analyze the assay results. The analytical system may vary depending on the type of the detection signals used in the platforms. When fluorescence signals are used, the platforms are placed on a transparent substrate and the signals are read through a fluorescence microscope or a reader to extract information. In one embodiment of the present invention, the fluorescence signals may be generated by treating the platforms of the fiber bundle with a sample containing target analytes, allowing the detection substances to react specifically with the target analytes, and linking a fluorescent marker to the detection substances. In this embodiment, the fluorescence signals can be generated from the fiber strands reacted with the target analytes and coding information corresponding to the reactive fiber strands can be read to analyze the assay results. When the bioassay platforms are treated with a sample containing target analytes, the target analytes react specifically with the detection substances immobilized on the reactive fiber strands. Then, the detection substances are allowed to react specifically with the target analytes and a fluorescent marker is linked to the detection substances. As a result, the reaction signals can be generated only from the reactive fiber strands linked to the target analytes.

When color signals are used, the cross-sections are imaged using a general camera or cell phone camera under appropriate illumination and the intensities of signals can be extracted. In one embodiment of the present invention, the color signals may be generated by treating the platforms of the fiber bundle with a sample containing target analytes, allowing the detection substances to react specifically with the target analytes, and linking an enzyme capable of changing the color of the substrate to the detection substances. In this embodiment, the color signals can be generated only from the fiber strands reacted with the target analytes and coding information corresponding to the reactive fiber strands can be read to analyze the assay results.

The method of the present invention may employ any general bioassay process known in the art to generate signals for the detection of target analytes from the bioassay platforms. In the bioassay using the platforms, signals can be detected and analyzed without an additional dedicated system or analyzer for the platforms. This avoids the need for an expensive analytical system, and therefore, the coding process also contributes to a reduction in the costs associated with the platforms and the experimental analysis.

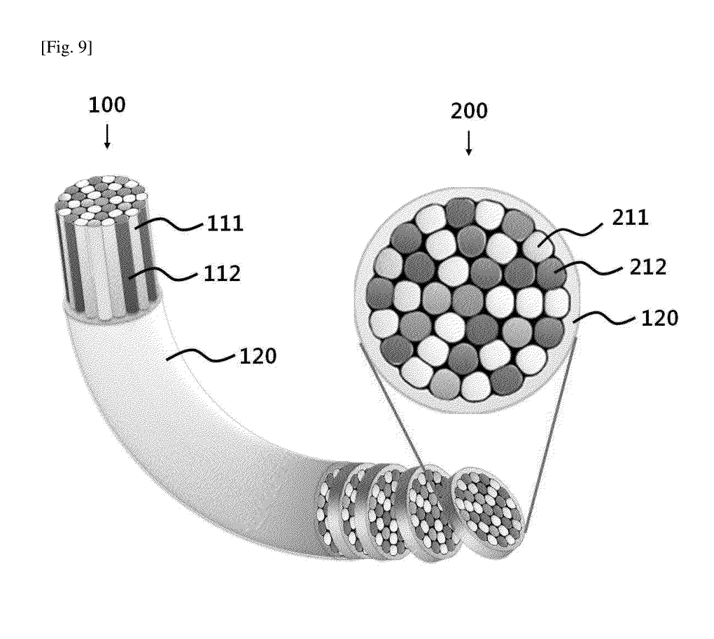

Another aspect of the present invention provides a bioassay platform including a matrix and a fiber bundle section composed of fragments of reactive fiber strands immobilized in the matrix wherein the fragments of the reactive fiber strands are coated with different types of detection substances. FIG. 9 is a design of a fiber bundle 100 and a bioassay platform 200 according to one embodiment of the present invention. Referring to FIG. 9, the bioassay platform 200 of the present invention may be fabricated by collecting reactive fiber strands 111 adsorbed by different types of detection substances and structuring the reactive fiber strands 111 with a matrix material 120 and may be in the form of a slice of the fiber bundle 100.

The multiplexed bioassay platform 200 of the present invention may include fragments 211 of the reactive fiber strands functionalized with various types of detection substances for the detection of target analytes. By the term “functionalized”, it is meant that specific detection substances are immobilized on the fiber strands to allow the fiber strands to have the function of reacting with target analytes. The number of kinds of analytes in bioassays can be increased simply by increasing the types and numbers of the fragments of the reactive fiber strands, making the platform highly scalable.

The multiplexed bioassay platform 200 of the present invention may have a shape in which the fragments 211 of the functionalized reactive fiber strands are structured by the matrix material 120. By this structuring, the fragments of the reactive fiber strands constituting the bioassay platform 200 can be spatially isolated from each other to prevent cross-contamination. The matrix material may include at least one material selected from the group consisting of adhesive films, polymer resins, and combinations thereof.

The multiplexed bioassay platform 200 of the present invention may further include fragments 212 of coding fiber strands including coding information related to information of the detection substances bound to the fragments of the reactive fiber strands. In one embodiment of the present invention, the coding information may include fluorescence signals or color signals. In one embodiment of the present invention, the coding fiber strands may be colored fiber strands. In FIG. 9, the fragments 211 of the reactive fiber strands are represented in white and the fragments 212 of the coding fiber strands are represented in various colors.

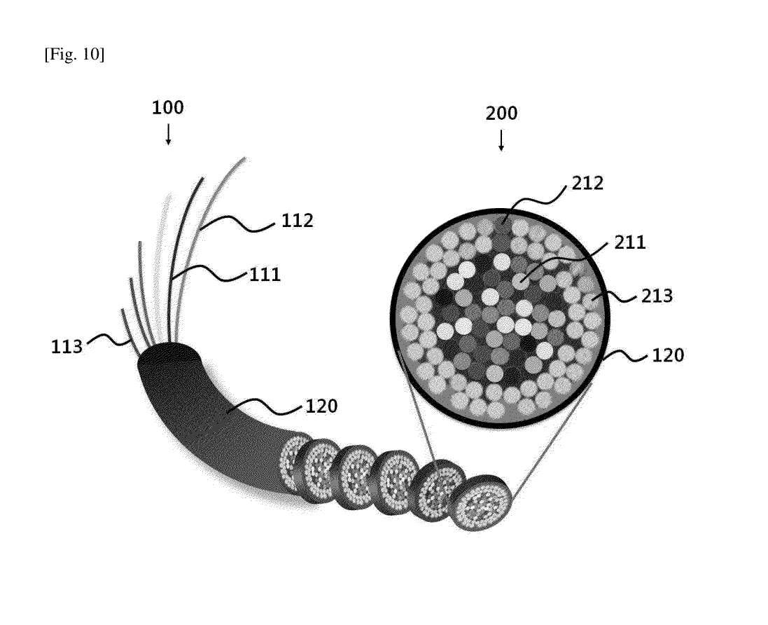

The multiplexed bioassay platform 200 of the present invention may further include fragments 213 of magnetically controllable fiber strands including a magnetic material. FIG. 10 is a design of a fiber bundle 100 and a bioassay platform 200 according to one embodiment of the present invention. In FIG. 10, fragments 211 of reactive fiber strands treated with various types of detection substances are represented in various colors and fragments 213 of magnetically controllable fiber strands are represented in green. In one embodiment of the present invention, the fragments 211 of the reactive fiber strands bound with various types of detection substances may be located at the central portion of the platform and the fragments 213 of the magnetically controllable fiber strands may surround the fragments 211 to form a control layer.

The multiplexed bioassay platform 200 of the present invention may further include control fiber strands. The control fiber strands may generate control signals between the reacted and unreacted fragments of the reactive fiber strands in bioassays.

The present invention will be explained with reference to the following examples.

### EXAMPLE 1

### Surface Treatment of Fiber and Coating with Reactive Substances

First, cotton threads were reacted with 5% of (3-aminopropyl)triethoxysilane (99%) in ethanol at 25° C. for 2 h and dried by annealing at 110° C. The threads were reacted with a solution of an anhydride (6% w/w) and triethylamine (0.84% v/v) diluted in amine-free dimethylformamide at 25° C. for 2 h, followed by washing. The threads were reacted with a solution of 5% N-hydroxysuccinimide and 5% ethyl(dimethylamino-propyl)carbodiimide diluted in MES buffer at 25° C. for 20 min to activate the functional groups. Finally, the activated thread strands were soaked into different solutions of individual capture antibodies as detection substances responding to three interleukins IL4, IL5, and IL7 as target analytes diluted in MES buffer and dried overnight, completing the production of three types of reactive fiber strands in which the individual capture antibodies responding to the interleukins were chemically bound to the thread strands.

### EXAMPLE 2

### Structuring of the Fiber and Formation of Slices

The reactive fiber strands treated with the three types of capture antibodies as biomolecules were located on transparent scotch tape in order, covered with another layer of scotch tape, and rolled up to make a fiber bundle. The fiber bundle was dipped in paraffin, which had been previously dissolved in a microwave oven, taken out from the paraffin, and cooled to room temperature to solidify the paraffin. The long fiber bundle covered with the paraffin was thinly cut into slices with constant thicknesses using a razor. The fragments of the reactive fiber strands performing their own functions were observed in the cross-sections of each slice. The slices were used as multiplexed bioassay platforms.

### EXAMPLE 3-1

### Bioassays Using Fluorescence Signals

Bioassays were conducted on the reactive fiber strands produced in Example 1 and the bioassay platforms fabricated in Example 2 as shown in Table 1.

TABLE 1

| | | | Amount per | | |
|---|---|---|---|---|---|
| Assay process | Reactive substances | Concentration | antibody | Time | Temperature |
| Immobilization of | Capture antibodies | 10 μg/ml | 15 μl | 2 h | 25° C. |
| capture antibodies | of IL4, IL5, IL7 | | | | |
| Selective antigen | IL4, IL5, IL7 | 10 μg/ml | 30 μl | 2 h | 4° C. |
| detection | | | | | |
| Binding of detection | Detection antibodies | 10 μg/ml | 30 μl | 1 h | 25° C. |
| antibodies | of IL4, IL5, IL7 | | | | |
| Binding of fluorescent | Enzyme labeled with | 5 μg/ml | 30 μl | 30 min | 25° C. |
| material | fluorescent material | | | | |

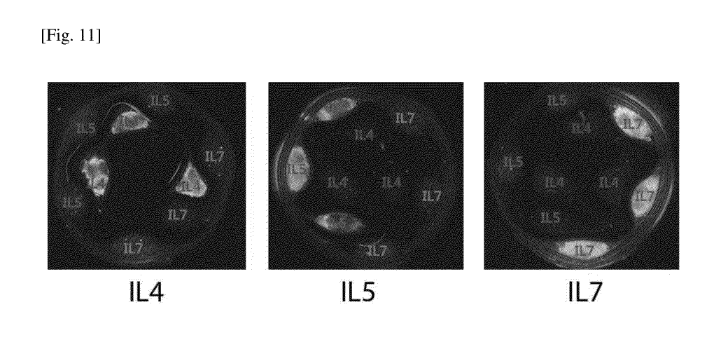

FIG. 11 shows the results of bioassays for interleukin 4, interleukin 5, and interleukin 7 using the platforms. The fiber bundle sections composed of the fragments of the reactive fiber strands treated with various types of capture antibodies were treated with interleukin 4, interleukin 5, and interleukin 7 as antigens. As a result, only the fiber strands functionalized with the capture antibodies responding to the antigens emitted fluorescence and the fluorescence signals could be detected based on the positional information of the fragments of the reactive fiber strands in the fiber bundle sections.

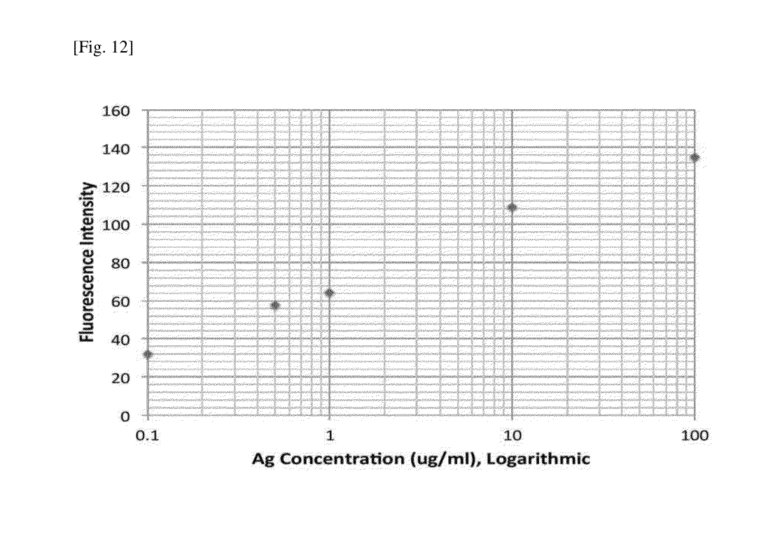

FIG. 12 is a graph showing a change in fluorescence intensity depending on the antigen concentration. The limit of detection was 10 pg/ml. The signal intensity increased steadily with increasing interleukin concentration from 100 pg/ml to 100 μg/ml, confirming that the proteins can be quantified within this range.

### EXAMPLE 3-2

### Bioassays Using Color Signals

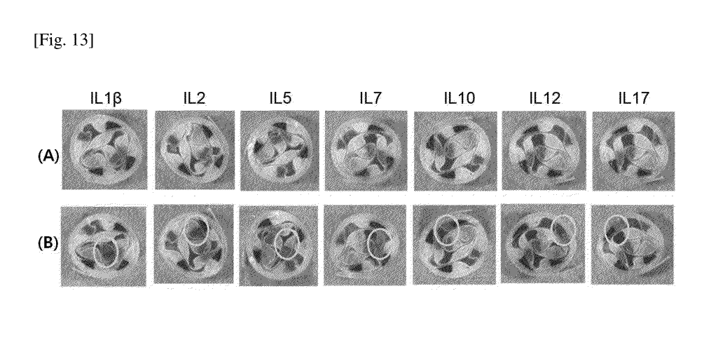

Individual capture antibodies responding to seven different types of interleukins (IL1beta, IL2, IL5, IL7, IL10, IL12, and IL17) were diluted in MES buffer, and then the fiber strands surface treated in Example 1 were soaked into the solutions and dried overnight, completing the production of seven types of functional reactive fiber strands in which the individual capture antibodies responding to the interleukins were chemically bound to the fiber strands. Then, the reactive fiber strands treated with the seven types of capture antibodies as biomolecules and a fiber for control signals were paired with eight different color coding fibers and the pairs were taped (IL1beta reactive fiber/red coding fiber, IL2 reactive fiber/orange coding fiber, IL5 reactive fiber/yellow coding fiber, IL7 reactive fiber/green coding fiber, IL10 reactive fiber/blue coding fiber, IL12 reactive fiber/brown coding fiber, IL17 reactive fiber/violet coding fiber, and control fiber/black coding fiber). In the same manner as the structuring process in Example 2, the eight pairs of fibers were rolled up using scotch tape to make fiber bundles, covered with paraffin, and thinly cut using a razor to fabricate bioassay platforms (see (A) of FIG. 13).

Bioassays were conducted on the platforms as shown in Table 2.

TABLE 2

| | | | Amount per | | |
|---|---|---|---|---|---|
| Assay process | Substances | Concentration | antibody | Time | Temperature |
| Immobilization of | Capture antibodies of | 10 μg/ml | 15 μl | 2 h | 25° C. |
| capture antibodies | IL1beta, IL2, IL5, IL7, | | | | |
| | IL10, IL12, IL17 | | | | |
| Selective antigen | IL1beta, IL2, IL5, IL7, | 10 μg/ml | 30 μl | 2 h | 4° C. |
| detection | IL10, IL12, IL17 | | | | |
| Binding of detection | Detection antibodies of | 10 μg/ml | 30 μl | 1 h | 25° C. |
| antibodies | IL1beta, IL2, IL5, IL7, | | | | |
| | IL10, IL12, IL17 | | | | |
| Enzyme binding | Enzyme | 5 μg/ml | 30 μl | 30 min | 25° C. |
| Signal amplification | Substrate | — | 15 μl | 8 min | 25° C. |

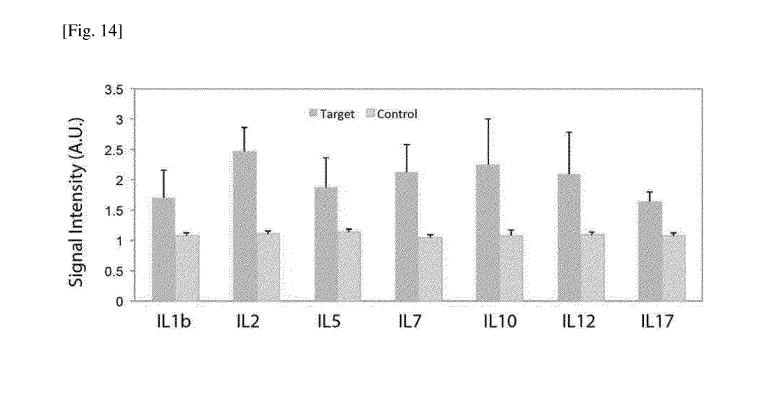

The platforms were reacted with individual liquids including the seven types of interleukins as antigens. After washing, each reaction product was reacted with a detection antibody solution for detecting the specific interleukin to attach the detection antibody thereto. The antibody-attached product was reacted with an enzyme solution to attach the enzyme thereto. The enzyme was used to determine whether the detection antibody was attached. Finally, the enzyme-attached product was reacted with a substrate solution reacting with the enzyme and a change in the color of the fiber from white to blue was observed. The cross-sections of the fragments where color signals were generated were observed visually or by microscopy or were imaged using a general camera and color-changed portions of the reacted fibers and information of the coding fibers were read from the image to analyze the assay results. FIG. 13 shows camera images of the cross-sections of the platforms (A) before and (B) after assays for the seven kinds of interleukins. Referring to FIG. 13, the signals could be distinguished through the colors of the coating fiber strands and blue color signals were observed only in the fragments of the reactive fiber strands on which the interleukins were immobilized. FIG. 14 is a graph showing the signal intensities during the bioassays. Compared to the control fiber (yellowish green bars), the fibers (targets, violet bars) responding to the target interleukins showed high signal intensities.

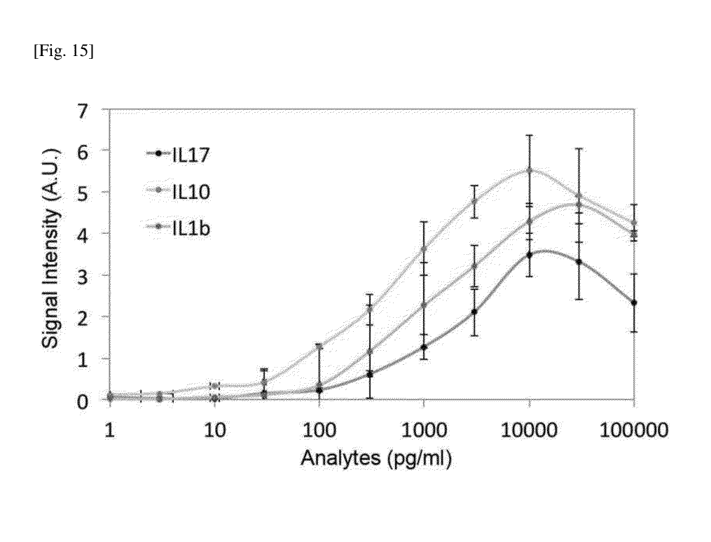

FIG. 15 shows changes in the intensity of color signals depending on the concentrations of three interleukins (ILlbeta, IL10, and IL17) as analytes. The limit of detection was 10 pg/ml. The signal intensities increased steadily with increasing interleukin concentrations from 100 pg/ml to 10 μg/ml, confirming that the proteins can be quantified in this range.

### EXAMPLE 3-3

### Multiplexed Bioassays Using Color Signals

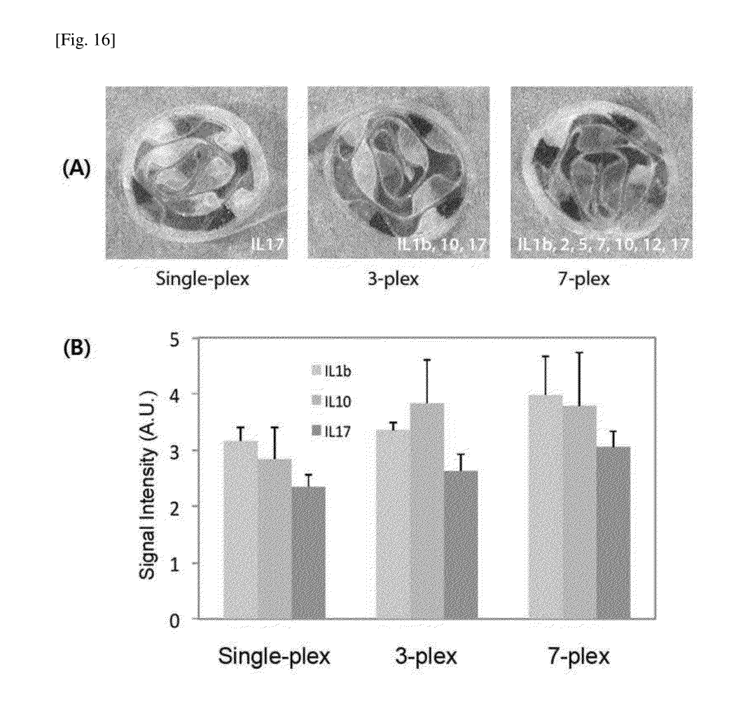

Multiplexed bioassays were conducted on the platforms fabricated in Example 3-2. The assays were conducted as shown in Table 2, except that the platforms were simultaneously treated with various kinds of antigens in the antigen immobilization step. FIG. 16 shows the bioassay results. Specifically, FIG. 16 (A) shows camera images of the platform treated with interleukin 17 as an antigen (Single-plex), the platform simultaneously treated with three interleukins (IL1beta, IL10 and IL17) (3-plex), and the platform treated with all seven interleukins (IL1beta, IL2, IL5, IL7, IL10, IL12, and IL17) (7-plex). FIG. 16(B) is a graph showing the signal intensities in the assays. The individual target analytes could be detected even when the platforms were simultaneously treated with the target analytes. It was found that the detection signals had high intensities even in the multiplexed assays.

1. A method for fabricating bioassay platforms, comprising: (a) coating the surfaces of fiber strands with different types of detection substances reacting specifically with target analytes to obtain reactive fiber strands, (b) collecting and structuring the reactive fiber strands coated with the detection substances to form a fiber bundle wherein the reactive fiber strands are immobilized using a matrix material in the course of the structuring, and (c) cutting the fiber bundle such that the cross-sections lie perpendicular to the lengthwise direction of the fiber bundle, to obtain slices in which fragments of the fiber strands are immobilized in the matrix material.

2. The method according to claim 1, wherein the detection substances are coated by physical adsorption or chemical binding.

3. The method according to claim 1, wherein the structuring further comprises arranging coding fiber strands to identify information of the detection substances present in the reactive fiber strands.

4. The method according to claim 3, wherein the structuring is performed by varying the color, number, arrangement order, size or a combination thereof of the coding fiber strands corresponding to the reactive fiber strands.

5. The method according to claim 1, wherein the structuring further comprises arranging magnetically controllable fiber strands into which a magnetic material is introduced.

6. The method according to claim 1, wherein the fiber is a cellulose-based fiber.

7. The method according to claim 1, wherein the fiber strands are spatially separated from each other in the fiber bundle formed by the structuring.

8. A bioassay method comprising: (a) coating the surfaces of fiber strands with different types of detection substances reacting specifically with target analytes to obtain reactive fiber strands, (b) collecting and structuring the reactive fiber strands coated with the detection substances to form a fiber bundle wherein the reactive fiber strands are immobilized using a matrix material in the course of the structuring, (c) cutting the fiber bundle such that the cross-sections lie perpendicular to the lengthwise direction of the fiber bundle, to obtain slices in which fragments of the fiber strands are immobilized in the matrix material, and (d) using the slices as bioassay platforms.

9. The bioassay method according to claim 8, wherein the assay results are evaluated through detection signals labeled on the cross-sections of the fiber bundle sections.

10. The bioassay method according to claim 9, wherein the detection signals are obtained from at least one piece of information selected from the group consisting of the positions of the reactive fiber strands in the fiber bundle, markers linked to the target analytes, and coding fiber strands structured with the reactive fiber strands in the fiber bundle.

11. A bioassay platform comprising: a matrix and a fiber bundle section composed of fragments of reactive fiber strands immobilized in the matrix wherein the fragments of the reactive fiber strands are coated with different types of detection substances.

12. The bioassay platform according to claim 11, wherein the matrix material is selected from the group consisting of adhesive films, paraffin, polymer resins, and combinations thereof.

13. The bioassay platform according to claim 11, wherein the fiber bundle section further comprises fragments of coding fiber strands comprising coding information related to information of the detection substances.

14. The bioassay platform according to claim 13, wherein the coding information may comprise fluorescence signals or color signals.

15. The bioassay platform according to claim 11, wherein the fragments of the reactive fiber strands constituting the fiber bundle section are spatially isolated from each other to prevent cross-contamination.

16. The bioassay platform according to claim 11, wherein the fiber bundle section further comprises fragments of magnetically controllable fiber strands comprising a magnetic material.

17. The bioassay platform according to claim 11, wherein the matrix comprises a magnetic material.
